SEQUENCE LISTING (2 sequences)

SEQ ID NO: 1 - protein, 4 aa, Artificial Sequence
  Synthetic Peptide
WMDF

SEQ ID NO: 2 - protein, 5 aa, Artificial Sequence
  Synthetic Peptide
AWMDF

(19) United States
(12) Patent Application Publication
(10) Pub. No.: US 2006/0172004 A1
(43) Pub. Date: Aug. 3, 2006

(54) Oral compositions for the stimulation of gastric acid secretion comprising pentagastrin

(76) Inventor: Sabina Glozman, Rehovot (IL)

Correspondence Address: FENNEMORE CRAIG, 3003 NORTH CENTRAL AVENUE, SUITE 2600, PHOENIX, AZ, 85012, US

(21) Appl. No.: 11/378,187

(22) Filed: Mar. 17, 2006

Related U.S. Application Data
(63) Continuation of application No. PCT/IB04/03076, filed on Sep. 21, 2004.
(60) Provisional application No. 60/505,159, filed on Sep. 24, 2003.

(51) Int. Cl. A61K 38/17; A61K 9/48; A61K 9/20

(52) U.S. Cl. 424/451; 424/464; 514/12

(57) ABSTRACT

The present invention discloses oral compositions comprising pentagastrin (PG) as an effective diagnostic agent for gastric acid secretion. The composition further comprises one or more agents that preserve the availability of PG in the gastric fluids, so that the biological activity of PG is maintained. The pharmaceutical compositions according to the present invention can be applied as diagnostic agents in the determination of the maximum gastric acid secretion as well as therapeutics.

### RELATED APPLICATION DATA

This application is a continuation of PCT/IB2004/003076 filed on Sep. 21, 2004, which is based on and claims the benefit of U.S. Provisional Application No. 60/505,159 filed on Sep. 24, 2003, which is incorporated herein in its entirety by this reference.

### FIELD OF THE INVENTION

The present invention relates to an effective gastric secretion stimulator administered orally for diagnosing diseases associated with abnormal gastric acid secretion.

### BACKGROUND OF THE INVENTION

Assessment of gastric acid secretion may play an important role in the diagnosis of a number of disease states in the gastrointestinal tract. These conditions include situations in which acid secretion is abnormally low such as atrophic gastritis. In such diseases, stimulation of acid secretion may serve to prove or refute dysfunction of the gastric mucosa. In other situations such as incomplete vagotomy following surgery for peptic disease, or uncontrolled upper gastrointestinal bleeding, relative hypersecretion of gastric acid may occur. In such cases, stimulation of gastric acid secretion may serve to test the potential for secretion of gastric acid. The assessment of acid secretion may also be important in conditions which are caused by mucosal damage secondary to gastric acid. These include for example Zollinger/Ellison syndrome (ZES), gastroesophageal reflux disease (includes esophagitis) and peptic ulcer diseases.

At present, the investigation of acid secretion in the stomach is carried out with the aid of chemical stimulation. Chemical secretion stimulators known in the art include histamine, gastrin-analogue preparations and histalog (an analog of histamine). The selectivity of histamine is limited, so that in addition to gastric secretion, it produces a number of side effects in the patient, such as reddening of the skin, nausea, vomiting, headache, dizziness, bronchospasm, edema of the rima vocalis, hypotension, and shocks. In most cases, these side effects can be fully eliminated by introducing such traditional antihistamines as H₁-antagonists, including mepyramine, tavelgil and suprastin. For this reason, histamine is used for what is known as the histamine test only in hospitals and in a limited number of cases.

Another known stimulant of gastric acid secretion is pentagastrin (PG) which is a pentapeptide containing the carboxyl terminal tetrapeptide of gastrin. This carboxyl terminal tetrapeptide is the active portion found in essentially all natural gastrins. In animals, PG acts to induce gastric acid secretion mainly via induction of histamine release from enterochromafin-like (ECL) cells residing in the stomach. The release of histamine and the consequent activation of histamine receptors residing on the parietal cells, leads to the activation of the parietal cells to actively secrete proton ions to the gastric lumen. It is also possible that PG acts directly on the parietal cells to induce its activation.

The low solubility of PG in acidic environment and the fact that PG is prone to pepsin degradation in the stomach, rendered its use as an inducer of gastric acid secretion following oral administration clearly unexpected until Applicants discovery. Prior to Applicants discovery, PG was considered by anyone skilled in the art to only be effectively active at inducing acid secretion if administered via parenteral routes. This factor hampers routine application of said compounds because such injections require sterile instruments and qualified staff in specialized centers. Indeed, no effect on acid secretion was noted in four normal subjects subjected to oral administration of PG, whereas some effect was noted in three additional patients with gastrointestinal abnormalities (Morrell & Keynes Lancet. 1975; 2(7937):712). In fact, this study was cited in a pharmacology textbook as a proof of lack of PG activity when administered orally (Martindale Thirty-second edition, p1616, the Chapter: “Supplementary Drugs and Other Substances”). Furthermore, in vitro studies in which a bullfrog model was used, suggested that PG did not affect the gastric mucosa when applied to the luminal surface (Ayalon A. et al., 1981 The Am. J. Surg. 141:94-97).

The development of an effective inducer of gastric acid secretion for diagnostic purposes which may be administered orally would fulfill a long-felt need. None of the existing art discloses or suggests that PG may be used as an effective diagnostic agent following oral administration.

### SUMMARY OF THE INVENTION

It is an object of the present invention to provide an oral composition comprising a stimulator of gastric acid secretion, for evaluating the potential of gastric acid secretion and for diagnosing a subject suffering from abnormally high or low gastric acid secretory state.

The present invention is related to oral compositions for the stimulation of gastric acid secretion comprising a pharmaceutically effective amount of a peptide comprising the amino acid sequence Trp-Met-Asp-PheNH₂ (denoted as SEQ ID No. 1) as an activator of parietal cells. Preferred peptides to be used in the present invention are pentagastrin (PG) and/or a PG analogue. The composition further comprises one or more agents that preserve the availability of PG in the gastric fluids, so that the biological activity of PG is maintained thus enabling PG to act locally in the stomach.

The present invention is based on the inventors surprising discovery that PG is active locally when administered orally, preferably by acting locally in the gastric lumen to activate the acid secreting cells. The oral compositions of the present invention are advantageous over the known gastric acid secretion stimulants. The present compositions permit activation of the parietal cells by PG without any side effects associated with systemic administration of PG due to the local effect of PG in the gastric lumen.

In a preferred embodiment, the oral compositions according to the present invention comprise PG or a PG analogue as a local activator of parietal cells in the gastric lumen. In addition to PG that comprises the amino acid sequence βAla-Trp-Met-Asp-PheNH₂ (SEQ ID NO:2), this invention contemplates the use of gastrin or PG analogues or derivatives thereof as parietal cell activators. Such variants include, but are not limited to the 34-, 17-, and 14-amino acid species of gastrin, and other truncation variants comprising the active C-terminal tetrapeptide of gastrin Trp-Met-Asp-PheNH₂ (SEQ ID NO:1), which is reported in the literature to have full pharmacological activity (see Tracey and Gregory (1964) Nature (London), 204: 935).

Also included are variants of gastrin and/or truncated gastrins where native amino acids are replaced with conservative substitutions. Various analogues of these molecules are also included, for example, but not limited to the N-protected derivative of PG Boc-βAla-Trp-Met-Asp-PheNH₂ in which Boc is tert-butyloxycarbonyl group or F-Moc-βAla-Trp-Met-Asp-PheNH₂ in which Moc is methoxycarbonyl.

In a non-limiting embodiment, the oral compositions according to the present invention further comprise one or more agents that preserve the availability of PG or its analogues in the acidic gastric fluids. These agents preferably are in an amount sufficient to preserve the availability of PG in the gastric fluids by retaining the solubility of PG in the gastric fluids and preventing its degradation, so that the local biological activity of PG in the stomach is preserved. This enables PG to act locally in the stomach to activate the parietal cells. Such agents are preferably antacids or alkaline agents that when dissolved in the gastric juice are capable of temporally elevating the pH of the gastric fluids to a value in which pepsin is inhibited, thereby inhibiting the degradation of PG in the gastric fluids by pepsin. Since PG is soluble only in alkaline conditions, the temporal elevation of the pH in the gastric fluids ensures that at least significant proportion of PG remains soluble in the gastric fluids.

It is noted that any weak or strong base (and mixtures thereof) can be utilized as the alkaline agent in the present oral compositions. The alkaline agent or the antacid is present in the composition in an amount sufficient to substantially preserve the stability and the solubility of PG in the acidic gastric fluids. Therefore, the alkaline agent of the present invention, when dissolved in the gastric juice, is capable of elevating the pH of the stomach to a value sufficient to achieve adequate availability of PG to effect therapeutic action.

According to a preferred embodiment, the alkaline agent in the composition is present in an amount sufficient to elevate the pH of the gastric fluids to a value above 4, and more preferably above 5, for a time period sufficient for PG to reach and activate the parietal cells in the stomach. In more preferred embodiment, the alkaline agent is capable of elevating the pH of the gastric fluids to a value above 5 for a time period ranging from 5 to 60 minutes, preferably for a time period ranging from 5 to 30 minutes. Thus, the alkaline agent according to the present invention preserves the solubility of PG in the gastric fluids for a time period sufficient for PG to activate the parietal cells. Furthermore, the temporal alkali condition in the gastric fluid prevents the degradation of PG by pepsin that is active only in acidic pH.

According to various embodiments, the present compositions further comprise other agents that preserve the availability of PG or its analogues in the acidic gastric fluids. Such agents are for example pepsin inhibitors (i.e., pepstain and its derivative bacitracin-cyclic dodecapeptide) that reduce the degradation of the peptide in the stomach or mucolytic agents that reduce the viscosity of the gastric mucosa, thereby accelerating the ability of PG to reach the cells responsible for acid secretion. Such mucolytic agents are for example reducing agents such as N-acetyl cysteine, dithiothreitol, citric acid or mannitol. The present compositions may further comprise an antibiotic effective against bacteria residing in the stomach.

In a preferred embodiment, the present oral composition comprising a gastric secretion stimulating effective amount of PG. Such oral composition contains the gastric secretion stimulator in immediate or sustained release form. The oral composition may be in the form of tablets, capsules, troches, lozenges, aqueous or oily suspensions, dispersible powders or granules, emulsions, multiparticulate formulations, syrups, elixirs and the like.

The present oral composition may be provided as a kit containing PG in one dosage form and the agent that preserves the availability of PG in the gastric fluids in a separate dosage form.

According to various embodiments, the oral composition further comprises excipients such as a filler, a lubricant, an agent for enhancing bio-adhesion of PG or its analogues in the stomach and a solubilizer. The release of PG in the stomach may be controlled and prolonged by means of controlled release, preferably gastro-retentive agents.

The oral compositions for the stimulation of gastric acid secretion according to the present invention may be used for diagnostic and therapeutic purposes. Specifically, the compositions according to the present invention may be used for diagnosing a subject suffering from a pathology associated with abnormal gastric acid secretion, for determining the extent of gastric acid secretion in a subject, for evaluating the efficacy of acid secretion inhibitors such as proton pump inhibitors and as a therapeutic composition in subjects in which stimulation of gastric acid secretion is required.

Thus in one embodiment, the present invention relates to a method of diagnosing a subject suffering from a disorder associated with abnormal gastric acid secretion, preferably abnormally high gastric acid secretion, the method comprising:
(a) administering to the subject a pharmaceutically effective amount of the oral composition according to the present invention to induce gastric acid stimulation; and
(b) determining the level of gastric acid secretion in said subject, wherein if the level of gastric acid secretion in said subject is greater or lower than the level determined in control subjects, than said subject suffers from a disorder associated with abnormal gastric acid secretion.

The method of diagnosing a subject suffering from a disorder associated with abnormal gastric acid secretion may further comprise the step of determining a baseline level of gastric acid secretion in the subject prior to step (a) and evaluating the extent of gastric acid secretion in said subject following the stimulation relative to the baseline secretion level.

The method of diagnosing a subject suffering from a disorder associated with abnormal gastric acid secretion may further comprise the step of administering to said subject a pharmaceutically effective amount of a proton pump inhibitor following step (b) and determining the level of gastric acid secretion following the administration of the proton pump inhibitor.

Preferred methods for determining the baseline gastric acid secretion level and the secretion level following stimulation involve for example measurements of the pH in the gastric lumen or measurements of total acid output in the gastroduodenal lumen.

Preferred peptide to be used in the methods of the present invention is pentagastrin (PG) (denoted as SEQ ID No. 2). However, this invention contemplates the use of other gastrin or PG analogues or derivatives thereof.

These methods may be used to screen patients for diseases associated with abnormal gastric acid secretion. The methods may also be used for monitoring the efficacy of medications administered to patients suffering from diseases associated with abnormal gastric acid secretion such as proton pump inhibitors and H₂ blockers. In one embodiment, such pathologies include diseases associated with abnormally high gastric acid secretion. These methods may also be used to screen patients for diseases associated with abnormal low gastric acid secretion such as atrophic gastritis.

The present invention further relates to a method for stimulating gastric acid secretion in a subject, the method comprising administering to the subject the oral composition according to the present invention to induce gastric acid stimulation. Preferred peptide to be used in the method of the present invention is PG (denoted as SEQ ID No. 2). However, this invention contemplates the use of other gastrin or PG analogues or derivatives thereof.

The methods of the present invention are effective in diagnosing or inducing gastric acid secretion in a mammal. Such mammal is for example a rodent, bovine, horses, canine, equine, non-human primate or human. According to a preferred embodiment, said mammal is human.

These and further embodiments will be apparent from the detailed description and examples that follow.

### BRIEF DESCRIPTION OF THE FIGURES

FIG. 1 shows that intragastric administration of PG to rats is effective in decreasing gastric pH in both intact (A) and pylorus-ligated rats (B).

FIG. 2 demonstrates the effect of oral PG on gastric acid output in pylorus-ligated rats (A) and the dose dependency of the effect (B).

FIG. 3 demonstrates the activity of different batches of PG in stimulating acid secretion.

FIG. 4 demonstrates the effect of tetragastrin administered per os as compared to subcutaneous injection on gastric pH (A) and acid output (B) in pylorus-ligated rats.

FIG. 5 demonstrates that NaHCO₃ preserves PG stability in artificial gastric fluid.

FIG. 6 demonstrates the percentage of non-degraded PG in various pH values.

### DETAILED DESCRIPTION OF THE INVENTION

The term “alkaline agent” refers to any pharmaceutically appropriate weak base or strong base (and mixtures thereof) that, when formulated or delivered with (e.g., before, during and/or after) PG, functions to temporally elevate the pH in the gastric lumen to a value that substantially preserves the availability of PG in the stomach.

The term “an agent that preserves the availability of PG in the stomach” refers to any agent that is capable of maintaining the solubility and stability of PG in the stomach. Specifically, such an agent is capable of maintaining at least a substantial amount of PG in a soluble form and non-degraded in the gastric juice, so that the biological activity of PG in the stomach is maintained.

The term “biological activity of PG in the stomach” refers to its activation of parietal cells located in the gastric lumen.

The present invention discloses for the first time that it is not necessary to apply PG via the parenteral route in order to induce gastric acid secretion. Accordingly, PG administered orally may be used as an effective diagnostic agent for gastric acid secretion. The pharmaceutical compositions according to the present invention can be applied as diagnostic agents in the determination of the maximum gastric acid secretion. In addition, the present pharmaceutical compositions may be used for therapeutic purposes, for example for treating diseases in which stimulation of gastric acid secretion is required.

The Applicants have unexpectedly discovered that the addition of an alkaline agent such as NaHCO₃ in a concentration sufficient to elevate the pH above 5.0 prevented the degradation of PG by pepsin. In vivo experiments also revealed that PG administered orally induced gastric acid secretion in an animal model. Furthermore, the in vivo experiments revealed that PG may act locally on the gastric wall, rather than through its absorption into the blood stream. Therefore, PG administered orally is capable of exerting a local effect in the stomach.

As disclosed herein, PG administered orally in the form of a tablet or solution is effective in inducing gastric acid secretion. Thus, it is possible to carry out both sub-maximum and maximum acid secretory tests using PG administered orally. Oral PG administration for the diagnosis of gastric acid secretion has a number of important advantages over the preparations used in the art (parenteral histamine or PG). The most important feature is that it may be administered orally, and it is highly selective, safe and convenient for use under clinical conditions. The administration of PG is not followed by any significant side effects typical of the histamine test; therefore, it is unnecessary to introduce any antihistamine preparations in advance. Moreover, oral application of the drug is predicted to cause even less side effects as compared to the parenteral administration of PG.

The peptide used in the present invention for inducing gastric acid secretion comprises the amino acid sequence βAla-Trp-Met-Asp-PheNH₂ (denoted as SEQ ID No. 2). However, other gastrin or PG analogues, or derivatives thereof are within the scope of the present invention. Such analogues or derivatives are well known to those of skill in the art. Such variants include, but are not limited to the 34-, 17-, and 14-amino acid species of gastrin, and other truncation variants comprising the active C-terminal tetrapeptide Trp-Met-Asp-PheNH₂ denoted as SEQ ID No. 1 which is reported in the literature to have full pharmacological activity (see Tracey and Gregory (1964) Nature (London), 204: 935).

Also included are variants of gastrin and/or truncated gastrins where native amino acids are replaces with conservative substitutions. Also include are various analogues of these molecules, including for example, but not limited to the N-protected derivatives of PG. Suitable protecting groups for PG include standard hydroxylprotecting groups known in the art, e.g., methoxymethyl (MOM), β-methoxyethoxymethyl (MEM), triakylsilyl, triphenylmethyl (trityl), tert-butoxycarbonyl (t-BOC), ethoxyethyl (EE), f-MOC (methoxycarbonyl), TROC, etc. The protecting group(s) may be removed by using standard procedures which are generally known to those skilled in the art to give the desired PG derivatives (T. W. Green, Protective Groups in Organic Synthesis, Chapter 2, pages 10-69 (1981)).

Gastrins, pentagastrins, or analogues thereof are commercially available. In addition, synthetic protocols are well known. Thus, for example, PG can be chemically synthesized using well-known peptide synthesis methodologies (see, e.g. Barany and Merrifield Solid-Phase Peptide Synthesis; pp. 3-284 in The Peptides: Analysis, Synthesis, Biology. Vol. 2: Special methods in peptide synthesis, part a.; Merrifield et al. (1963) J. Am. Chem. Soc., 85: 2149-2156; and Stewart et al. (1984) Solid Phase Peptide Synthesis, 2nd ed. Pierce Chem. Co., Rockford, ILL.). Additionally, PG can be chemically synthesized, for example, by conjugation of a Boc-Ala residue to the tetrapeptide Trp-Met-Asp-PheNH₂.

Oral formulations of PG are effective in diagnosing the extent of gastric acid secretion and in determining the maximum gastric acid secretion ability. The oral PG formulations may be used to screen patients for diseases associated with abnormal gastric acid secretion. The oral formulations of PG may also be used for monitoring the efficacy of medications aimed to reduce gastric acid secretion such that are administered to patients suffering from diseases associated with damage secondary to gastric acid secretion. Such pathologies include for example diseases such as reflux esophagitis, gastritis, duodenitis, gastric ulcer and duodenal ulcer.

Furthermore, the oral formulations of PG of the present invention may be used to screen patients for diseases of other gastrointestinal disorders with abnormal high gastric acid secretion, e.g. in patients on nonsteroidal anti-inflammatory drugs (NSAID) therapy (including low dose aspirin), in patients with Non Ulcer Dyspepsia, in patients with symptomatic gastro-esophageal reflux disease (GERD), in patients with gastrinomas, in patients with acute upper gastrointestinal bleeding, in conditions of stress ulceration. Further, the oral formulations of PG may be used for screening Helicobacter infections and diseases related to these. Other conditions well suited for screening according to this invention include, but are not limited to Zollinger-Ellison syndrome (ZES), Werner's syndrome, and systemic mastocytosis.

The compositions of the present invention comprise PG or an analog thereof in an effective amount to achieve a pharmacological effect on the parietal cells without undue adverse side effects. The standard approximate amount of PG present in the compositions is preferably in an amount of 1-100 mg, more preferably 2-60 mg, and most preferably 4-40 mg of PG (or an equivalent amount of a PG analogue).

The amount of PG administered in the present compositions is sufficient to cause a measurable increase in gastric acid secretion, more preferably to cause a significant increase in gastric acid secretion (e.g., a statistically significant increase at the 90%, more preferably at the 95%, and most preferably at the 98% or 99% confidence level).

In a preferred embodiment, the compositions of the present invention further comprise one or more agents that preserve the availability of PG in the acidic gastric fluids. More specifically, the preservation agent maintains the stability or the solubility of PG in the gastric fluids. This enables PG to act locally in the stomach to activate the parietal cells. Such agents are preferably alkaline agents or antacids that when dissolved in the gastric juice are capable of elevating the pH of the gastric fluids to a pH in which the gastric-residing peptidases are inhibited and at least significant proportion of PG remains soluble in the gastric fluids.

Alkaline agents to be used in the present invention include for example: sodium or potassium bicarbonate, magnesium oxide, hydroxide or carbonate, magnesium lactate, magnesium glucomate, aluminum hydroxide, aluminium, calcium, sodium or potassium carbonate, phosphate or citrate, di-sodium carbonate, disodium hydrogen phosphate, a mixture of aluminum glycinate and a buffer, calcium hydroxide, calcium lactate, calcium carbonate, calcium bicarbonate, and other calcium salts. It is noted that while sodium bicarbonate dissolves easily in water, calcium carbonate is water-insoluble and is slowly soluble only in acidic environment. Therefore, calcium carbonate may be useful when sustained dissolution of the alkaline agent in the stomach is desired.

Examples of antacids to be used in the present invention include one or more of the following: alumina, calcium carbonate, and sodium bicarbonate; alumina and magnesia; alumina, magnesia, calcium carbonate, and simethicone; alumina, magnesia, and magnesium carbonate; alumina, magnesia, magnesium carbonate, and simethicone; alumina, magnesia, and simethicone; alumina, magnesium alginate, and magnesium carbonate; alumina and magnesium carbonate; alumina, magnesium carbonate, and simethicone; alumina, magnesium carbonate, and sodium bicarbonate; alumina and magnesium trisilicate; alumina, magnesium trisilicate, and sodium bicarbonate; alumina and simethicone; alumina and sodium bicarbonate; aluminum carbonate, basic; aluminum carbonate, basic, and simethicone; aluminum hydroxide; calcium carbonate; calcium carbonate and magnesia; calcium carbonate, magnesia, and simethicone; calcium carbonate and simethicone; calcium and magnesium carbonates; magaldrate; magaldrate and simethicone; magnesium carbonate and sodium bicarbonate; magnesium hydroxide; magnesium oxide.

Preferably, the compositions of the present invention comprise one or more alkaline agents or antacids in an effective amount to achieve a pharmacological effect. Specifically, the alkaline agents or antacids in the composition are present in an amount sufficient to elevate the pH of the gastric fluids to a pH above the pH optima for proteases found in the stomach for a time period sufficient for PG to activate the parietal cells in the stomach. In a preferred embodiment, the alkaline agents or antacids are present in an amount sufficient to elevate the pH of the gastric fluids to a pH above 5 for a time period ranging from 5 to 60 minutes, preferably for a time period ranging from 5 to 30 minutes. The quantity of alkaline agents required in the compositions of the present invention will necessarily vary with several factors including the type of alkaline agent used and the equivalents of base provided by a given alkaline agent. In practice, the amount required to provide good availability of PG in the stomach is an amount which, when added to a solution of 200 milliliters of artificial gastric fluid (prepared according to the United States Pharmacopea (USP) guideline), raises the pH of that HCl solution to at least pH 5.0. Preferably, at least 100 milligrams, and more preferably at least 300, and most preferably at least 500 milligrams of the alkaline agents are used in the pharmaceutical compositions of the invention.

In another embodiment, the compositions of the present invention further comprise other agents that preserve the availability of PG in the acidic gastric fluids. For example, the compositions may comprise pepsin inhibitors such as the activated pentapeptide pepstatin and its derivatives, either of natural or synthetic origin. These inhibitors might decrease the degradation of PG by pepsin. Furthermore, the compositions may comprise mucolytic agents that reduce the viscosity of the gastric mucosa, thereby accelerating the ability of PG to reach the parietal cells. Such mucolytic agents are for example reducing agents such as N-acetyl cysteine, dithiothreitol, citric acid or mannitol. Alternatively, the compositions may also comprise a polymeric coating for PG, such as, an enteric-coating polymers to protect the PG from the acidic environment of the stomach.

The pharmaceutical compositions containing the PG are administered in oral dosage forms. Such oral dosage forms contain the drug in immediate or sustained release form and suitable pharmaceutically acceptable carriers. The compositions of the present invention may be formulated in either solid or liquid form. It is noted that solid formulations are preferred in view of the improved stability of solid formulations as compared to liquid formulations. The oral dosage forms may be in the form of tablets, capsules, troches, lozenges, aqueous or oily suspensions, dispersible powders or granules, emulsions, multiparticulate formulations, syrups, elixirs, and the like.

PG and the one or more agents that preserve the availability of PG in the gastric fluids are preferably formulated in a single solid dosage form such as multi-layered tablets, suspension tablets, effervescent tablets, powder, pellets, granules or capsules comprising multiple beads. In another embodiment, the active agents may be formulated in a single liquid dosage form such as suspension containing all active ingredients or dry suspension to be reconstituted prior to use.

The active ingredients of the present invention may be incorporated within inert pharmaceutically acceptable beads. In this case, the drug(s) may be mixed with further ingredients prior to being coated onto the beads. Ingredients include, but are not limited to, binders, surfactants, fillers, disintegrating agents, alkaline additives or other pharmaceutically acceptable ingredients, alone or in mixtures. Binders include, for example, celluloses such as hydroxypropyl methylcellulose, hydroxypropyl cellulose and carboxymethyl-cellulose sodium, polyvinyl pyrrolidone, sugars, starches and other pharmaceutically acceptable substances with cohesive properties. Suitable surfactants include pharmaceutically acceptable non-ionic or ionic surfactants. An example of a suitable surfactant is sodium lauryl sulfate.

The particles may be formed into a packed mass for ingestion by conventional techniques. For instance, the particles may be encapsulated as a “hard-filled capsule” using known encapsulating procedures and materials. The encapsulating material should be highly soluble in gastric fluid so that the particles are rapidly dispersed in the stomach after the capsule is ingested.

In another embodiment, the active ingredients of the present invention are packaged in compressed tablets. The term “compressed tablet” generally refers to a plain, uncoated tablet for oral ingestion, prepared by a single compression or by pre-compaction tapping followed by a final compression. Such solid forms can be manufactured as is well known in the art. Tablet forms can include, for example, one or more of lactose, mannitol, corn starch, potato starch, microcrystalline cellulose, acacia, gelatin, colloidal silicon dioxide, croscarmellose sodium, talc, magnesium stearate, stearic acid, and other excipients, colorants, diluents, buffering agents, moistening agents, preservatives, flavoring agents, and pharmaceutically compatible carriers. The manufacturing processes may employ one, or a combination of, four established methods: (1) dry mixing; (2) direct compression; (3) milling; and (4) non-aqueous granulation. Lachman et al., The Theory and Practice of Industrial Pharmacy (1986). Such tablets may also comprise film coatings, which preferably dissolve upon oral ingestion or upon contact with diluent.

In another alternative, the compositions of the present invention are formulated in compressed forms, such as suspension tablets and effervescent tablets, such that upon reaction with water or other diluents, the aqueous form of the composition is produced for oral administration. These forms are particularly useful for medicating children and the elderly and others in a way that is much more acceptable than swallowing or chewing a tablet. The present pharmaceutical tablets or other solid dosage forms disintegrate alkaline agent with minimal shaking or agitation.

The term “suspension tablets” as used herein refers to compressed tablets which rapidly disintegrate after they are placed in water, and are readily dispersible to form a suspension containing a precise dosage of the PG and the alkaline agent. In one non-limiting example, the suspension tablets may comprise 4-20 mg PG and about 1-4 grams of sodium or calcium bicarbonate as an alkaline agent. To achieve rapid disintegration of the tablet, a disintegrant such as Croscarmellose sodium may be added to the formulation. The disintegrant may be blended in compressed tablet formulations either alone or in combination with microcrystalline cellulose, which is well known for its ability to improve compressibility of difficult to compress tablet materials. Microcrystalline cellulose, alone or co-processed with other ingredients, is also a common additive for compressed tablets and is well known for its ability to improve compressibility of difficult to compress tablet materials. It is commercially available under the Avicel trademark.

The suspension tablet composition may, in addition to the ingredients described above, contain other ingredients often used in pharmaceutical tablets, including flavoring agents, sweetening agents, flow aids, lubricants or other common tablet adjuvants, as will be apparent to those skilled in the art. Other disintegrants, such as crospividone and sodium starch glycolate may be employed, although croscarmellose sodium is preferred.

In addition to the above ingredients, the oral dosage forms described above may also contain suitable quantities of other materials, e.g. diluents, lubricants, binders, granulating aids, colorants, flavorants and glidants that are conventional in the pharmaceutical art. The quantities of these additional materials will be sufficient to provide the desired effect to the desired formulation. Specific examples of pharmaceutically acceptable carriers and excipients that may be used to formulate oral dosage forms are described in the Handbook of Pharmaceutical Excipients, American Pharmaceutical Association (1986), incorporated by reference herein.

In certain conditions, it is desirable to prolong the retention time of PG in the stomach in order to prolong the effect of PG in the lumen by using gastric retention formulations. Such formulation may be for example a formulation that unfolds rapidly within the stomach to a size that resists gastric emptying. Such systems retain their integrity for an extended period and will not empty from the stomach at all until breakdown into small pieces occurs. Caldwell et al. (Caldwell, L. J., Gardener, C. R., Cargill, R. C. (1988), U.S. Pat. No. 4,767,627) disclose a cross shaped device made of erodible polymer and loaded with drug which is folded and inserted into a hard gelatin capsule. Following oral administration the gelatin shell disintegrates and the folded device opens out. With a minimum size of 1.6 cm and a maximum size of 5 cm it will not pass from the stomach through the pylorus until the polymer erodes to the point where the system is sufficiently small that it can be passed from the stomach.

An alternative approach to prolong the retention time of PG in the stomach is to use a hydrophilic erodible polymer system such as Poly(ethylene oxide) (Polyox) and Hydroxypropyl-methylcellulose (HPMC) that is of a convenient size for administration to humans. On imbibing fluid the system swells over a short period of time to a size that will encourage prolonged gastric retention, allowing sustained delivery of contained drug to absorption sites in the upper gastrointestinal tract. Because these systems are made of an erodible and hydrophilic polymer or polymer mixture, they readily erode over a reasonable time period to pass from the stomach. The time period of expansion is such that this will not occur in the esophagus and if the system passes into the intestine in a partially swollen state, the erodibility and elastic nature of the hydrated polymer will eliminate the chance of intestinal obstruction by the device.

An alternative approach to preserve the availability of PG in the stomach is based on formation of emulsions or microemulsions containing PG. A microemulsion is a liquid dispersion of oil in water, stabilized by surfactants. The microemulsion particles are smaller than those of an emulsion, rendering the microemulsion essentially optically clear. It is generally believed that microemulsions are micelle-like particles, having an essentially micellar structure but containing a distinct oil phase in the micelle “core”. As a result, the hydrophobic therapeutic agent in a microemulsion-based delivery system is preferentially solvated in the triglyceride phase, which is in turn encapsulated in the swollen micelle. The preferential partitioning in the triglyceride phase results in higher loading capacities than in comparable micelle-based systems, but at the cost of introducing into the delivery system the lipolysis-dependence and other disadvantages associated with the presence of triglycerides. In addition, the larger size of microemulsion particles, relative to true micelles, results in a slower rate of particle diffusion, and thus a slower rate of therapeutic agent absorption.

The following examples are presented in order to more fully illustrate certain embodiments of the invention. They should in no way, however, be construed as limiting the broad scope of the invention. One skilled in the art can readily devise many variations and modifications of the principles disclosed herein without departing from the scope of the invention.

### EXAMPLES

### Example 1

### Orally-Administered PG is Effective in Stimulating Gastric Acid Secretion In Vivo

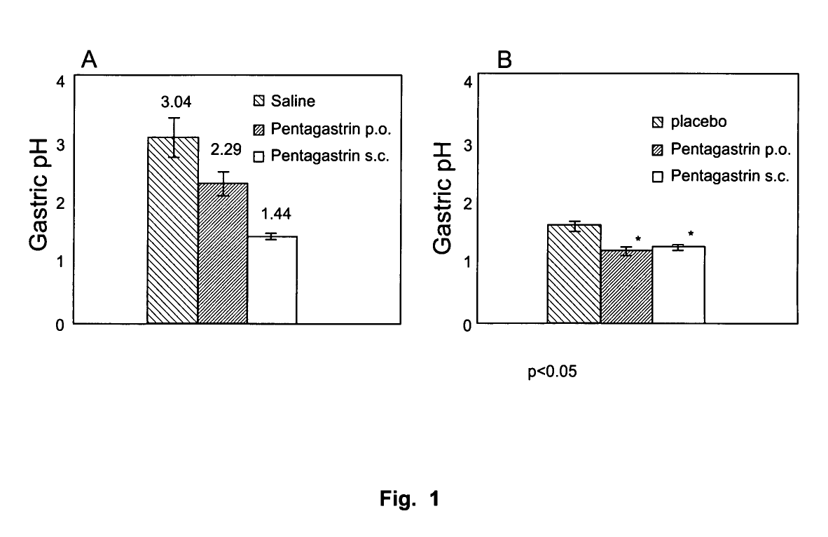

Experiments were undertaken to determine the bioactivity of orally administered PG on gastric acid secretion. As shown in FIG. 1A, the intragastric administration of PG (200 μg/kg) to anesthetized rats resulted in a decrease of gastric pH indicating that PG stimulated acid secretion. This effect was observed at 10 min following PG administration (data not shown) and reached its maximal level 30 min after oral administration of PG (p=0.053). Subcutaneous (S.C) administration of PG (6 μg/kg) served as a positive control and resulted in a decrease of gastric pH (p=0.01). This observation suggested that PG administered orally was bioactive in the stomach.

The observation raised the question whether PG exerts its effect on acid secretion by direct action on gastric mucosa or whether it reaches the gastric mucosa via the blood stream following its absorption from the small intestine (the site through which most proteins are normally absorbed). To differentiate between these two possibilities, the effect of PG on acid secretion was tested following oral administration to rats in which the pylorus was ligated. As demonstrated in FIG. 1B, PG (250 μg/kg) decreased gastric pH in pylorus-ligated rats, indicating that PG acted directly within the stomach.

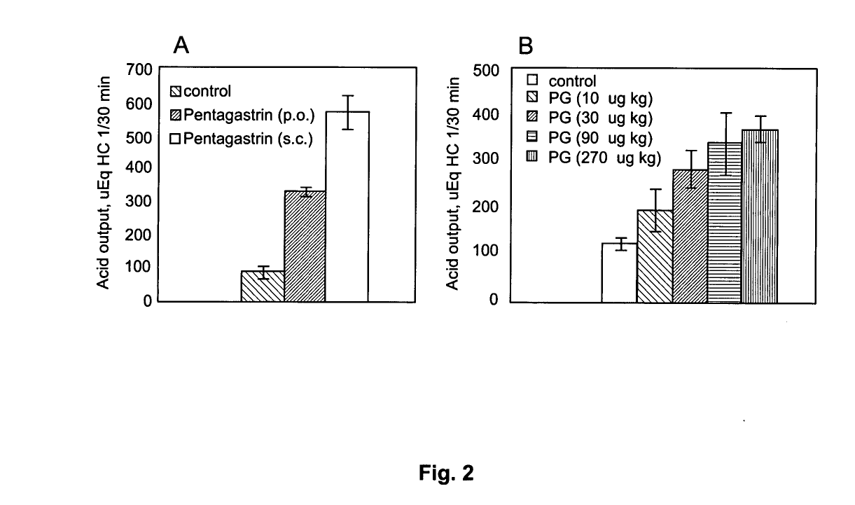

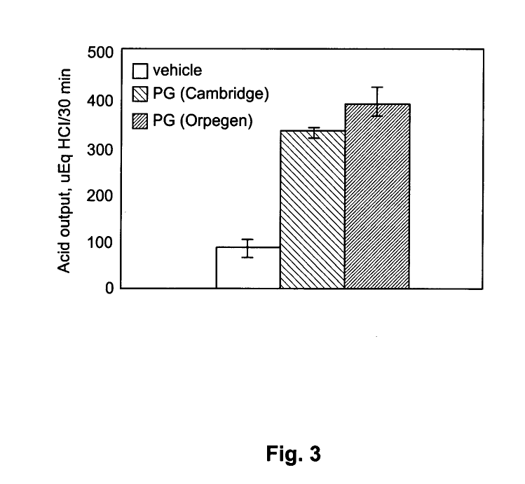

FIG. 2 demonstrates the effect of oral PG on gastric acid output in pylorus-ligated rats (A) and the dose-dependent effect of orally administered PG on gastric acid secretion (B). The activity of different batches of PG in stimulating acid secretion is further demonstrated in FIG. 3.

### Example 2

### Orally-Administered Tetragastrin is Effective in Stimulating Gastric Acid Secretion in Rat

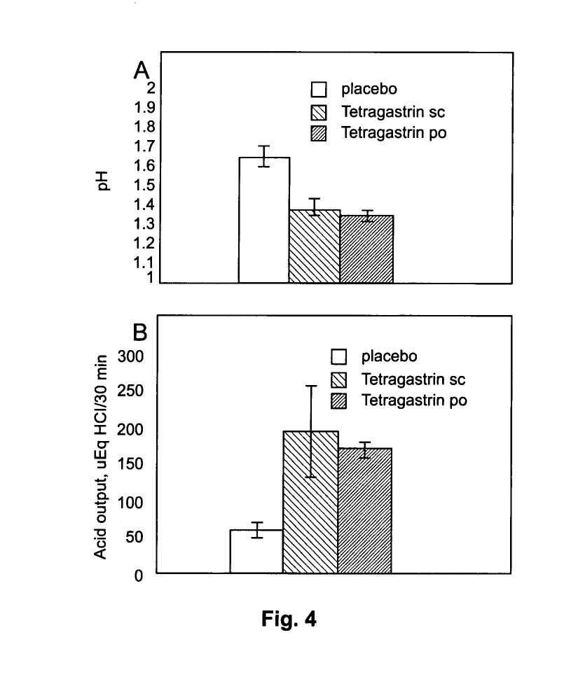

The effect of tetragastrin (Trp-Met-Asp-PheNH₂) administered orally in stimulating gastric acid secretion was examined. FIG. 4 demonstrates that tetragastrin administered either subcutaneously (s.c) or orally into Wister rats reduced gastric pH (A) and elevated acid secretion (B). Tetragastrin was administered either s.c. (25 μg/kg) or per os (206 μg/kg) and gastric juice was collected 30 min following treatment. Acid concentration was measured by titration the samples with NaOH to pH=7. Results are expressed as means±SEM of 3-4 animals. Similarly to PG, equimolar dose of tetragastrin led to a local stimulation of gastric acid secretion, although this effect was less prominent as compared to PG.

### Example 3

### NaHCO₃ Preserves PG Stability in Artificial Gastric Fluid

The stability of PG in acidic pH in the presence of NaHCO₃ was tested in vitro using artificial gastric fluid. Artificial gastric fluid was prepared in accordance with U.S. Pharmacopoeia (USP) 2000 Ed., P. 235. For preparing 200 ml of gastric fluid, 0.4 g of NaCl and 0.64 g of Pepsin were dissolved in 16 ml 1M HCl and 184 ml of water. The pH of the gastric fluid was 1.2. Ten or twenty ml of 8.4% (1M) NaHCO₃ (final concentration 3.72 mg/ml or 7.12 mg/ml, respectively) and 16 ml of 250 ppm PG solution (0.25 mg/ml) were added to the solution. The concentration of PG in the final solution was 16 ppm. When indicated, Omeprazole granules were added as well (solutions B and C). In order to determine the stability of PG in the final solution over time, HPLC analysis was performed on samples taken at the following time points post preparation: 0′ (immediately following preparation), 5′, 10′, 20′, 40′, 60′. To stop the reaction, the pH was adjusted to 7.5-8.5 using NH₄OH.

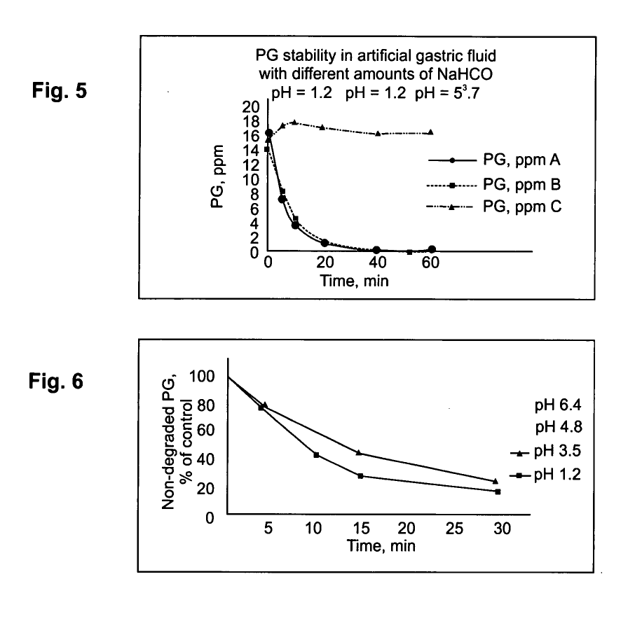

As demonstrated in FIG. 5, fast degradation of PG was observed in solutions A and B that comprise PG in the presence of 3.72 mg/ml of NaHCO₃ (pH 1.2). However, PG remained stable for 1 h in solution C that comprises 7.12 mg/ml of NaHCO₃ (pH 5.7). These results indicate that the addition of an alkaline agent such as NaHCO₃ in a concentration sufficient to elevate the pH above 5.0 prevents the degradation of PG by pepsin. FIG. 6 further demonstrates that at least 80% of PG remains non-degraded for at least 15 min in pH 4.8.

### Example 4

### Lack of Systemic Absorption of PG Following Oral Administration of ³H-Labeled PG

The bioavailability of PG in rats following oral administration of ³H-labeled PG was studied. Male Wister Hanover rats were anaesthetized and catheters were positioned in the jugular vein for blood sampling. Rats were treated orally with ³H-PG at a dose of 50 μg/kg (50×10⁶ CPM per dose) and blood samples were drawn at 5, 15 and 30 minutes. The radioactivity in blood samples at each time point was determined by a β-counter.

TABLE 1

| | Radioactivity in systemic circulation |
|---|---|
| Time following oral administration | % of Total Radioactivity/Dose |
| 5 min. | 1.43 |
| 15 min. | 1.67 |
| 30 min. | 1.42 |

The data indicate that approximately 1.5% (748×10³, 982×10³ and 832×10³ CPM at 5, 15 and 30 min, respectively) of the total radioactivity (50×10⁶ CPM) that was administered orally entered the systemic circulation. This observation suggests that the radioactivity corresponds to tritiated amino acids and not to tritiated-PG, since time-dependent degradation kinetics is expected for the intact PG. These data suggest that oral administration of PG is likely to result in local activity due to rapid degradation and low systemic absorption.

### Example 5

### The Effect of a CCK-B Antagonist on PG-Mediated Gastric Acid Secretion in Rats

As PG is a gastrin hormone homologue, its local effect is thought to be mediated via gastrin pathway, i.e. an activation of gastrin receptor (CCKB). To test this hypothesis the effect of the specific CCKB antagonist (Itriglumide) on PG-mediated acid secretion in rats was examined.

In this study, rats were anesthetized with Ketamine and Domitor mixture and provided with 20 mg/kg of Itriglumide that was administered intraduodenally (i.d.). Following 15 min, gastric pylorus was ligated and 300 μg/kg PG was administered into the stomach (i.g.). After 30 min, gastric juice was obtained, centrifuged and the volume and pH of the supernatants were measured. The acid concentration (titratable acidity) was analyzed by titration the gastric juice samples with NaOH and total acid output expressed in pEq HCl was calculated by multiplying the sample volume by the acid concentration. As revealed from the results presented in Table 2 below, intraduodenal injection of CCKB antagonist (ant.) inhibits the local effect of PG on gastric acid secretion in rats.

TABLE 2

Acid Output

| Group | MEAN | ±SEM |
|---|---|---|
| PG (i.g.), 300 ug/kg | 60.056 | 10.43 |
| CCKB ant. (i.d.) 20 mg/kg | 15.24 | 2.82 |
| Placebo of PG (i.g.) - NH4HCO3 | 19.25 | 3.03 |
| Placebo of CCKB ant. - saline (i.d.) | 12.93 | 1.55 |
| PG (i.g.), 300 ug/kg and | 22.884 | 2.70 |
| CCKB ant.(i.d.) 20 mg/ml | | |
| PG (i.g.), 300 ug/kg and | 51.74 | 9.35 |
| Placebo of CCKB ant. - saline (i.d.) | | |

Student t-test

| | |
|---|---|
| PG vs. ant. + PG | P = 0.0023 |
| | P = 0.0042 |
| | P = 0.0016 |

### Example 6

### The Effect of Intraduodenal Injection of PG on Acid Secretion in Anesthetized Pylorus-Ligated Rats

The effect of intraduodenal injection of PG on acid secretion in anesthetized pylorus-ligated rats was examined. In this study, 300 μg/kg PG was administered intraduodenaly in anesthetized pylorus-ligated rats and the level of gastric acid secretion was determined 30 minutes later. Gastric juice was obtained, centrifuged and the volume and pH of the supernatants were measured. The acid concentration (titratable acidity) was analyzed by titration gastric juice samples with NaOH and total acid output expressed in μEq HCl was calculated by multiplying the sample volume by the acid concentration. As a control the equal amount of PG was injected intragastrically and the effect of PG on gastric secretion was determined. As demonstrated in Table 3, both intragastric and intraduodenal injection of PG induce gastric acid secretion in anesthetized pylorus-ligated rats.

TABLE 3

Acid Output

| Group | MEAN | ±SEM |
|---|---|---|
| (i.g.), 300 ug/kg | 45.89 | 6.37 |
| Placebo (i.g.) | 12.46 | 2.65 |
| PG (i.d.), 300 ug/kg | 42.26 | 6.95 |
| Placebo (i.d.) | 11.65 | 1.44 |

Student t-test

| | |
|---|---|
| PG (i.g.) vs. Placebo | P = 0.000125 |
| | P = 0.000243 |
| | P = 1.981 × 10⁵ |

### Example 7

### Tablets Comprising PG, Sodium Bicarbonate and Calcium Carbonate

The tablets are formulated as a single dosage form in which each tablet containing the following ingredients:

| | |
|---|---|
| PG | 4 mg |
| NaHCO₃ | 500 mg |
| CaCO₃ | 500 mg |
| Croscarmellose sodium | |
| Microcrystalline cellulose (Avicel) | |
| Magnesium stearate | |
| Starch | |

### Example 8

### Effervescent Sacs Comprising PG and Sodium Bicarbonate

Effervescent tablets are formulated as a single dosage containing the following ingredients:

| | |
|---|---|
| PG | 4 mg |
| NaHCO₃ | 958 mg |
| Citric acid | 832 mg |
| Potassium carbonate | 312 mg |
| Magnesium stearate | |
| Starch | |

### Example 9

### Capsules Comprising Sucrose-PG Beads and Calcium Carbonate

Hard gelatin capsules are formulated as a single dosage form comprising mixed population of particles. Each capsule contains the following ingredients:

4 mg PG loaded on inert sugar beads

600 mg calcium carbonate (CaCO₃)

hydroxypropyl methylcellulose (HPMC)

The PG solution is sprayed on inert sugar pellets (Nu-Pareils, 25/30) in a fluidized bed apparatus. After drying, the PG-sugar beads are further coated with CaCO₃ and with hydroxypropyl methylcellulose (HPMC) to form the final PG particles. The final PG particles and calcium carbonate powder are packed into size 0 hard gelatin capsules in an amount corresponding to 4 mg PG and 600 mg calcium carbonate per capsule.

Upon dissociation of the gelatin capsules in the stomach, the PG beads are expanded due to the contact of the HPMC layer of the PG-containing beads with the gastric juice, thereby delaying the release of PG from the particles. The rate of PG release is determined by the thickness and the erosion rate of the HPMC layer of the PG beads.

It will be appreciated by a person skilled in the art that the present invention is not limited by what has been particularly shown and described hereinabove. Rather, the scope of the invention is defined by the claims that follow.

1. An oral pharmaceutical composition comprising as active ingredients: (i) a pharmaceutically effective amount of a peptide comprising the amino acid sequence of SEQ ID NO:1, and (ii) at least one agent that preserves the availability of the peptide in gastric fluids.

2. The oral composition of claim 1, wherein the peptide is pentagastrin (PG) having the amino acid sequence of SEQ ID NO:2 or a synthetic analog thereof.

3. The oral composition of claim 2, wherein the preservation agent is one or more alkaline agents, wherein the amount of the alkaline agent is sufficient to preserve the availability of PG in the stomach so that the biological activity of PG is maintained.

4. The oral composition of claim 3, wherein the alkaline agent is selected from the group consisting of: calcium carbonate, sodium or potassium bicarbonate, magnesium oxide, hydroxide or carbonate, magnesium lactate, magnesium glucomate, aluminum hydroxide, aluminium, calcium, sodium or potassium carbonate, phosphate or citrate, di-sodium carbonate, disodium hydrogen phosphate, a mixture of aluminum glycinate and a buffer, calcium hydroxide, calcium lactate, calcium carbonate and calcium bicarbonate.

5. The oral composition of claim 3, wherein the oral composition is formulated in a single unit dosage form and the alkaline agent is in an amount of at least 300 mg.

6. The oral composition of claim 2, wherein the peptide is in an amount sufficient to locally activate parietal cells located in the gastric lumen.

7. The oral composition of claim 6, wherein the amount of PG is between 2 to 60 mg.

8. The oral composition of claim 5, wherein the single unit dosage form is a compressed tablet, double-layered tablet, a press-coat tablet, a multi-particulate capsule, an effervescent tablet, a suspension tablet, solution, or suspension comprising PG particles and at least one alkaline agent.

9. The oral composition of claim 8, wherein the amount of the alkaline agent is sufficient to preserve the availability of PG in the stomach so that the biological activity of PG is maintained.

10. The oral composition of claim 8, wherein the PG particles are optionally coated with enteric-coating or with time-dependent release polymers.

11. The oral composition of claim 10, wherein the time-dependent release polymers comprise at least one polymer capable of swelling in aqueous environment.

12. The oral composition of claim 11, wherein the polymer capable of swelling in aqueous environment is selected from the group consisting of: a synthetic polymer and cellulose-based polymer, or substituted derivative thereof.

13. The oral composition of claim 1, wherein the composition further comprising a pepsin inhibitor, a mucolytic agent, a controlled release agent, a bioadhesive polymer or an antibiotic effective against bacteria residing in the stomach.

14. The oral composition of claim 2, wherein the peptide is the N-protected derivative of PG selected from the group consisting of: methoxymethyl (MOM), β-methoxyethoxymethyl (MEM), trialkylsilyl, triphenylmethyl (trityl), TIPSO, tert-butoxycarbonyl (t-BOC), ethoxyethyl (EE), F-MOC, and TROC.

15. An oral pharmaceutical kit comprising as active ingredients: (i) a pharmaceutically effective amount of a peptide comprising the amino acid sequence of SEQ ID NO:1; and (ii) at least one agent that preserves the availability of the peptide in the gastric fluids.

16. The kit of claim 15, wherein the active ingredients are formulated in separate unit dosage forms.

17. A method for stimulating gastric acid secretion in a subject in need thereof, the method comprising administering to the subject an oral pharmaceutical composition comprising as active ingredients: (i) a pharmaceutically effective amount of a peptide comprising the amino acid sequence of SEQ ID NO:1; and (ii) at least one agent that preserves the availability of the peptide in gastric fluids.

18. The method of claim 17, wherein the peptide is a fragment of gastrin or a synthetic analog thereof.

19. The method of claim 18, wherein the peptide is PG (denoted as SEQ ID No. 2), an active fragment thereof, or a synthetic analog of PG.

20. The method of claim 19, wherein the peptide is an N-protected derivative of PG selected from: methoxymethyl (MOM), -methoxyethoxymethyl (MEM), trialkylsilyl, triphenylmethyl (trityl), TIPSO, tert-butoxycarbonyl (t-BOC), ethoxyethyl (EE), F-MOC, and TROC.

21. The method of claim 17, wherein the subject is a human subject.

22. A method of diagnosing a subject suffering from a disorder associated with abnormal gastric acid secretion, the method comprising the following steps:
(a) administering to the subject an oral pharmaceutical composition comprising as active ingredients: (i) a pharmaceutically effective amount of a peptide comprising the amino acid sequence of SEQ ID NO:1, and (ii) at least one agent that preserves the availability of the peptide in gastric fluids, the composition stimulates gastric acid secretion, and
(b) determining the level of gastric acid secretion in said subject following the stimulation, wherein if the level of gastric acid secretion in said subject following the stimulation is greater or lower than the level determined in control subjects, the subject is determined to suffer from a disorder associated with abnormal gastric acid secretion.

23. The method of claim 22, wherein the peptide is a fragment of gastrin or a synthetic analog thereof.

24. The method of claim 23, wherein the peptide is PG (denoted as SEQ ID No. 2), an active fragment thereof, or a synthetic analog thereof.

25. The method of claim 24, wherein the peptide is an N-protected derivative of PG selected from: methoxymethyl (MOM), -methoxyethoxymethyl (MEM), trialkylsilyl, triphenylmethyl (trityl), TIPSO, tert-butoxycarbonyl (t-BOC), ethoxyethyl (EE), F-MOC, and TROC.

26. The method of claim 22, wherein the subject is a human subject.

27. The method of claim 22, wherein the disorder is associated with abnormally high gastric acid secretion.

28. The method of claim 27, wherein the disorder is selected from the group consisting of: reflux esophagitis, gastritis, duodenitis, gastric ulcer, duodenal ulcer, a pathology associated with nonsteroidal anti-inflammatory drugs (NSAID) therapy, non-ulcer Dyspepsia, gastro-esophageal reflux disease (GERD), gastrinomas, acute upper gastrointestinal bleeding, stress ulceration, Helicobacter infections, Zollinger-Ellison syndrome (ZES), Werner's syndrome and systemic mastocytosis.

29. The method of claim 22, further comprising determining a baseline level of gastric acid secretion in the subject prior to step (a) and evaluating the extent of gastric acid secretion in said subject following the stimulation relative to the baseline secretion level.

30. The method of claim 22, further comprising administering to said subject a pharmaceutically effective amount of a proton pump inhibitor following step (b) and determining the level of gastric acid secretion following the administration of the proton pump inhibitor.
